(19) United States
(12) Patent Application Publication
(10) Pub. No.: US 2022/0127572 A1
(43) Pub. Date: Apr. 28, 2022

(54) METHOD FOR LARGE-SCALE PRODUCTION OF ALLOSPECIFIC TYPE 1 REGULATORY TREGS (TR1) STABLE IN THE PRESENCE OF PROINFLAMMATORY CYTOKINES WITH THERAPEUTIC POTENTIAL IN TRANSPLANTATION

(71) Applicant: UNIVERSIDAD NACIONAL AUTONOMA DE MEXICO, Ciudad De Mexico (MX)

(72) Inventors: Maria Gloria SOLDEVILA MELGAREJO, Tlalpan (MX); Saul ARTEAGA CRUZ, Iztapalapa (MX); Evelyn Katy ALVAREZ SALAZAR, Milpa Alta (MX); Arimelek CORTES HERNANDEZ, Coyoacan (MX); Josefina ALBERU GOMEZ, Tlalpan (MX)

(21) Appl. No.: 17/510,902

(22) Filed: Oct. 26, 2021

(30) Foreign Application Priority Data
Oct. 26, 2020 (MX) MX/A/2020/011355

(51) Int. Cl. C12N 5/0783; A61K 9/00; A61K 39/00; A61P 37/06; C12N 5/0784

(52) U.S. Cl. CPC C12N 5/0637; A61K 9/0019; A61K 39/001; A61P 37/06; C12N 5/064; A61K 2039/515; C12N 2501/51; C12N 2501/2304; C12N 2501/231; C12N 2502/1121; C12N 2501/2302; C12N 2501/515; C12N 2501/22

(57) ABSTRACT

A methodology to obtain large numbers of allospecific human Tr1 lymphocytes in vitro differentiated with phenotype and suppressive function stability in presence of proinflammatory cytokines, by using donor tolerogenic dendritic cells (DC10) derived from donor monocytes and from a not related receptor (allogeneic) naîve T cells cocultures. The obtained cells with the present methodology are characterized by the expression of a Tr1 regulatory phenotype (CD4+, CD49b+, LAG-3+), being high IL-10 producers, and also they express additional co-inhibitory molecules as PD1, TIM-3, CD39, CTLA-4 y TIGIT. Moreover, the cellular product obtained by this methodology is able to maintain a stable phenotype and suppressive function in presence of proinflammatory cytokines (IL-1β, IL-6, IFN-γ y TNF-α). The numbers, purity, and stability of the Tr1 obtained by this methodology, make them great candidates for their use as therapeutic tools in transplantation.

### CROSS-REFERENCE TO RELATED APPLICATIONS

This application claims the benefit of Mexican Patent Application No. MX/a/2020/011355, filed Oct. 26, 2020, the contents of which are incorporated herein by reference.

### TECHNICAL FIELD OF THE INVENTION

The present invention provides a new method for the generation and expansion of allospecific human Type 1 regulatory T cells to induce tolerance to transplants and their use as an alternative or complementary therapy to conventional immunosuppression.

### BACKGROUND OF THE INVENTION

Transplantation is considered the most effective therapy for patients with advanced chronic injury in some organs. In the last 20 years, the quality and life expectancy of transplanted patients has increased due to the use of immunosuppressive drug-based therapy, whose purpose is to prevent allograft rejection development mediated by the adaptive and innate response from the patient immune system [1]. Currently, the survival rate of kidney transplants is above 90% at 5 years. However, long-term use of immunosuppressive drugs transplanted patients results in adverse events, including the development of cardiovascular diseases, increased susceptibility to infections, and neoplasia emergence, due to the widespread effect on the immunological system [2] [3] [4].

One of the most promising alternatives for tolerance induction to the allograft is Treg-based therapy, which includes the use of CD4+ T cells, aiming to specifically suppress inflammatory responses towards the graft and to maintain immune cell homeostasis [5] [6] [7].

The majority of Treg cells that have been used in immunotherapy for transplantation are denominated thymic regulatory T cells (tTreg), characterized by the expression of surface CD25, and the transcription factor Foxp3 [8]. These cells have been shown to be effective in the context of bone marrow allograft transplant to avoid graft versus host disease (GVDH). However, Foxp3+ Tregs have the disadvantage that they do not have a superficial specific marker that allows their purification prior to their transfusion into the patients [9] [10] [11]. Thus, in the last years, several research groups have proposed alternative therapies with another type of regulatory T cells denominated Type 1 regulatory T lymphocytes (Tr1).

Tr1 cells are CD4+ T lymphocytes capable of suppressing immune effector responses. They were shown to play an important role in oral tolerance and the intestinal microenvironment [12] [13]. Tr1 cells can be in vitro differentiated from CD4+ naïve T cells under suboptimal activating signals in the presence of anti-inflammatory cytokines [14] [15] [16]. This subpopulation is characterized by high production of IL-10, as well as TGF-β and a low or null IL-4 and IL-2 production [17] [18].

Tr1 lymphocytes were described for the first time in patients with Severe Combined Immunodeficiency (SCID) that had developed tolerance to allograft after allogeneic hematopoietic stem cell transplantation. These patients presented higher concentration levels of IL-10 in the serum, compared to those that had not developed allograft tolerance [19]. Later, it was demonstrated the presence of an alloreactive T lymphocyte subpopulation from which it was possible to identify, purify and clone the IL-10 coding gene, suggesting the development of tolerance mechanisms mediated by T lymphocytes [20].

Subsequent studies by Baccheta and coworkers described that this T CD4+ subpopulation had a low proliferative capability and high production of IL-10, when activated in vitro [21]. Further, it was demonstrated in a murine preclinical model of Inflammatory Bowel Disease (IBD), that in vitro stimulation of CD4⁺ T lymphocytes in the presence of exogenous IL-10, identified a T cell subpopulation that, when infused to IBD mice, was capable to resolve colitis, confirming the great potential of Tr1 cells in the regulation of inflammatory processes [20].

Importantly, Gagliani and coworkers reported the coexpression of the surface molecules CD49b and LAG-3 as specific markers of Tr1 subpopulation, both in murine and human CD4+T cells [22], allowing the identification and isolation of this T cell subset, which greatly contributed to the development of new protocols for their use in cellular therapy.

One of the first developed methodologies for the in vitro obtention of Tr1 lymphocytes was based on the coculture between peripheral blood mononuclear cells (PBMCs) or isolated CD4+ cells and monocytes (CD14+) obtained from different donors, in the presence of exogenous IL-10. The result was an heterogeneous population of anergic cells with suppressive capability against several antigens that contained Tr1 lymphocytes [14] [23].

Another developed methodology based on the coculture of isolated CD3+ and total PBMCs from 2 different individuals in the presence of IL-10 for 10 days generated a cellular product, which consisted of an heterogeneous population of anergic cells with suppressive capability, containing a low proportion of CD49b⁺LAG-3⁺ Tr1 lymphocytes (˜6% of the total population) and were named “IL-10 DLI” [24]. In the same work, it was reported clinical trial results from a cohort of 12 total patients with hematopoietic stem cell transplantation, using the IL-10 DLI population, to prevent the development of graft versus host disease (GVHD). Of note, four of those patients were able to avoid GVHD.

One of the most efficient methodologies for the generation of allospecific Tr1 cells is the use of tolerogenic DCs denominated DC10. These cells are characterized by the high production of IL-10, the expression of CD14, and the inhibitory receptors HLA-G and ILT4, which are crucial to allow the differentiation of allospecific Tr1 [25].

The tolerogenic capacity of DC10 was considered for the generation of regulatory T cells that were used in a large-scale collaborative international study for solid organ transplantation, named “the ONE Study” [26]. In this context, coculture between in vitro differentiated DC₁₀ from monocytes (CD14⁺) and isolated CD4+ T lymphocytes in the presence of exogenous IL-10 during 10 days, induces an heterogeneous population of anergic cells with suppressive capability that includes a percentage of Tr1 lymphocytes (6-12%), named “T10” was obtained [27]. This cellular product has been used for immunotherapy in patients with renal transplant [27].

Despite the progress reached in the design of new methodologies aimed to obtain Tr1 cells with therapeutic potential, the main focus of theses studies has been the large-scale obtention of anergic cells with suppressive capability with the goal to reach the requested number of cells for their infusion in patients (approximately 5×10⁵ to 2×10⁶ cells/kg), thereby sacrificing the purity of the Tr1 population transferred. Moreover, these studies have not included a detailed phenotypic characterization from their cellular products to ensure the obtention of an homogeneous population with stable regulatory functions. Moreover the reported methodologies have not achieved the sufficient numbers to represent a feasible method for the obtention of Tr1 on a large scale. In this context, recent studies have evidenced great heterogeneity among Tr1 cells (CD4⁺CD49b⁺LAG-3⁺), based on their IL-10 production, as well as differential co inhibitory molecule expression, including PD-1, TIM-3, CD39, CTLA-4 y TIGIT (“CIR phenotype”). Interestingly, IL-10⁺ CIR⁺ cells represent a subpopulation with the highest suppressive potential both in murine models and humans [28].

With the invention described here, we describe a new in vitro protocol to obtain a high yield of allospecific Tr1 lymphocytes from isolated naive CD4⁺ T lymphocytes, expressing an enriched CIR+ phenotype and which can be isolated and expanded on a large scale, maintaining their phenotype and suppressive function under proinflammatory environment.

### DESCRIPTION OF THE INVENTION

The present invention describes a new method for large-scale production of Tr1 cells (1000 times the initial number, reaching >5×10⁸ cells) for immunotherapy in transplantation, with high purity (>80%) through the isolation and in vitro expansion of allospecific Tr1 lymphocytes enriched in CIR phenotype and with stable suppressive function.

Based on the purity and yield obtained, our invention constitutes a feasible alternative to previously described methodologies for transplantation cell therapy. Also, with the established experimental conditions in the present invention, it is possible to obtain a population of human allospecific Tr1 lymphocytes with a phenotype CD49b⁺ LAG-3⁺ IL-10⁺ PD1⁺ CD39⁺ CTLA-4⁺ TIM-3⁺ TIGIT⁺, with a stable suppressive function in the presence of proinflammatory cytokines such as IL-1β, IL-6, IFN-γ, and TNF-α.

For the present invention, the terms “donor 1 and 2” refer to healthy donors, not genetically related (allogenic), so the compatibility grade among them is low or null. It is considered as “donor 3”, a non related individual different from donors 1 and 2.

The method to generate and expand in vitro allospecific Tr1 lymphocytes in the present invention considers four stages:
1. In vitro differentiation of tolerogenic dendritic cells, named DC₁₀, from donor 1.
2. Obtention of allospecific Tr1 lymphocytes by the coculture of allogeneic naive T cells (CD4⁺CD25⁻CD45RA⁺) from donor 2 peripheral blood, with DC₁₀ or DCs from donor 1 obtained in stage 1.
3. Isolation of Tr1 cells obtained in stage 2.
4. Polyclonal expansion of the cells from stage 3.

DC₁₀ differentiation is made from monocytes (CD14⁺), obtained from peripheral blood mononuclear cells (PBMCs) of donor 1 cultured for 7-8 days in RPMI medium with 10% of human serum (HS) AB (Gemini Bio Product) in the presence of human recombinant (hr) GM-CSF, hrIL-4 (50 ng/mL) and hrIL-10 (10 ng/mL). At this time, non-adherent cells, with large size and several projections in their membranes, can be identified, which express the surface molecules characteristic of DC₁₀ populations (CD14, HLA-G and ILT4).

The obtention of allospecific Tr1 lymphocytes was performed by co-culturing DC₁₀ from donor 1 and naive T lymphocytes from donor 2 which, in the context of transplantation, correspond to donor and recipient, respectively, in a 1:5 to 1:10 proportion. Naïve T cells were stained with the vital dye CellTrace™ Violet (CTV, Invitrogen), to evaluate T cell proliferation based on fluorescence loss. The differentiation culture is carried out in the presence of rhIL-10 (10 ng/mL) during 14 days, followed by a restimulation with rhIL-10 (10 ng/mL) and rhIL-2 (20-50 U/mL) on day 7 of culture. These conditions promote the differentiation and proliferation of allospecific Tr1 lymphocytes.

Isolation of Tr1 lymphocytes is performed based on the expression of CD4, CD49b, and LAG-3 from the proliferated allospecific CTV− population. Tr1 cells are purified by FACS sorting and then lymphocytes are expanded during 3 consecutive cycles, each one including an activation and resting phase. During the activation phase, Tr1 lymphocytes are cultured in the presence of anti-CD3/CD28-coupled beads in a proportion of 1:5 (beads:Tr1) in the presence of rhIL-10 (10 ng/mL) and hrIL-2 (200-250 U/mL) for 4 days. During the resting phase, the polyclonal activation stimulus is removed and Tr1 lymphocytes are cultured and maintained with rhIL-2 (20-50 U/mL) for 3 days to prevent induction of anergy (a hyporesponsive state) or activation-induced cell death. During the expansion cycles >80% of allospecific Tr1 cells maintain the expression of CD49b and LAG-3 and >90% show high production of IL-10, as well as expression of co-inhibitory receptors (>80%) involved in Treg suppressive function (CTLA-4, PD-1, CD39, TIGIT y TIM-3).

Tr1 cells were harvested in RPMI 1640 medium supplemented with FBS (20%), cells were centrifuged and the sample supernatant was discarded. Tr1 lymphocytes were resuspended in expansion medium supplemented with 10% of AB human serum (40-60×10³ per each 150-200 μL) in the presence of rhIL-2 (20-50 U/mL) and they were cultured in “U” bottom 96 well plates for 2-3 days.

The methodology developed in the present invention, allows the differentiation of up to 60% of allospecific Tr1 lymphocytes, surpassing the percentages reported by previously reported methodologies (˜6-12%). Furthermore, our protocol includes high purification of allospecific Tr1 lymphocytes (CD49b⁺LAG-3⁺) by flow cytometry, eliminating the potential heterogeneity in the cellular product.

The suppressive function of the cellular population obtained with the established conditions of the present invention, was evaluated by suppression assays which measure the capacity of allo-Tr1, to inhibit the proliferation of CD3⁺ T cells from donor 2, towards dendritic cells from donor 1 or from individuals genetically different to donors 1 and 2.

Notably, this invention allows the obtention of higher numbers of allospecific Tr1 lymphocytes, than those reported to be required in previous clinical assays (5×10⁵ to 2×10⁶ cells per kilogram patient), of which only ˜6-12% were Tr1[24] [27].

The present invention describes, for the first time, a methodology for obtaining Tr1 with 80% purity, which will allow reducing the number of cells required per kilogram body weight due to the cellular product characteristics.

Finally, in the present invention, allospecific Tr1 lymphocytes with a high phenotypic and functional stability have been obtained, even after an expansion in the presence of an inflammatory environment, thus providing greater safety for their use as cellular therapy.

### BRIEF DESCRIPTION OF THE FIGURES

FIG. 1A shows the co-expression of the HLA-G and ILT4 in conventional DCs (cDC) and DC10. DC10s contain a higher percentage of HLA-G+ ILT4+ cells compared to cDCs. As shown in the graph in FIG. 1B, expression levels of HLA-G and ILT4, calculated as mean fluorescence intensity are significantly higher in DC10 compared to cDC.

FIG. 2A of CD14 in cDCs and DC10, showing lower mean fluorescence intensity of this marker in DCs compared to DC10. FIG. 2B shows a graph of the difference between the CD14 mean fluorescence from cDCs and DC10, being significantly higher in the last.

FIG. 3A shows percentage of differentiated Tr1 cells on day 14 of co-cultures between naïve T cells and DC10 (Tr1) or naive T cells and cDCs (control) in presence or absence of rhIL-10, respectively. FIG. 3B shows a significant increase in the percentages of CD49b+LAG-3+ obtained under Tr1 culture conditions compared to control cultures.

FIG. 4A shows production of IL-10 from CD49b+LAG-3+ population on day 14 in the naive T:DCs (Control) and naive T:DC10, (Tr1) cocultures in presence or absence of rhIL-10 respectively. FIG. 4B shows a graph between the difference of IL-10 production in the T:DCs (control) and naive T:DC10, (Tr1) cocultures in presence or absence of rhIL-10 respectively, being significantly higher in the cultures with Tr1 differentiation conditions.

FIG. 5 The graph shows the relative increment on total allospecific Tr1 lymphocytes numbers obtained after 3 expansion cycles, reaching an increment of 1000× the initial Tr1 total cell numbers.

FIG. 6A shows coexpression of CD49b and LAG-3 at the end of the third expansion cycle shows the generation of an homogeneous Tr1 population. FIG. 6B graph shows a sustained expression of CD49b and LAG-3 in Tr1 lymphocytes during 3 expansion cycles.

FIG. 7A displays a representative experiment showing that nearly the entire Tr1 population is positive for IL-10 after the third expansion cycle. FIG. 7B graph shows the percentages of IL-10 production obtained after each expansion cycle, reaching values >90% in the third cycle.

FIG. 8A shows allospecific Tr1 populations show a highly suppressive phenotype, characterized by the expression of the co-inhibitory molecules PD1, TIM-3, CD39, CTLA-4 and TIGIT after the third expansion cycle. As shown FIG. 8B, Tr1 cells express high levels of all the co-inhibitory molecules evaluated in each expansion cycle.

FIG. 9A shows Tr1 lymphocytes present high suppressive function, evaluated by the decreased proliferation on allospecific CD4+ T cells, showing the highest suppression at a ratio 1:1 (naïve T cell:DC). In contrast, FIG. 9B shows no significant suppression was observed in the co-cultures stimulated with donor 3 DCs (third party), further confirming the allospecific suppressive function of Tr1 lymphocytes.

FIG. 10A shows Tr1 lymphocytes present a high suppressive function, evaluated by the decreased proliferation of allospecific CD8+ T cells, showing the highest suppression at a ratio 1:1 (naïve T cell:DC). In contrast, FIG. 10B shows no significant suppression was observed in the co-cultures stimulated with donor 3 DCs (third party), further confirming the allospecific suppressive function of Tr1 lymphocytes.

In FIG. 11A, it is observed that the percentage of CD49b and LAG-3 co-expression is not affected by the presence of proinflammatory cytokines, which provides the phenotype stability of allospecific Tr1 lymphocytes in the present invention. FIG. 11B shows a graph of the set of CD49b and LAG-3 coexpression in presence of all the proinflammatory cytokines separately or together, simulating a proinflammatory environment.

FIGS. 12A-12B shows a high and stable expression of the coinhibitory molecules TIM-3 (FIG. 12A) and TIGIT (FIG. 12B) in allospecific Tr1 cells, even after Tr1 lymphocytes were expanded in the presence of inflammatory cytokines separately or in combination.

FIG. 13. This Figure shows the percentage of Tr1 IL-10 producing cells obtained after the 3 additional expansion cycles in the presence of proinflammatory cytokines. A similar percentage of IL-10 production was observed in all cultures, both in the presence or absence of proinflammatory cytokines, except in those cultures stimulated with TNF-α (A).

FIG. 14. Tr1 lymphocytes preserve their suppressive functions, as evaluated by the inhibition of allospecific CD4+ T proliferation, even after being expanded and maintained in the presence of an inflammatory environment.

### EXAMPLES

### Example 1. Tolerogenic Dendritic Cells (DC10) Derived from CD14⁺ Monocytes

Buffy coat was obtained from blood samples of the National Institute of Respiratory Diseases blood bank (donor 1), and PBMCs were isolated by Ficoll-Paque™ Plus (GE Healthcare) density gradient, following the manufacturer's instructions. For all assays, PBMCs were cryopreserved in a solution containing 40% of RPMI 1640 (Gibco) media, 50% of fetal bovine serum (FBS, Bioswest), and 10% of Dimethyl Sulfoxide (DMSO, Gibco), in a cellular density of 10⁷ PBMCs/mL, for 24 hours at −70° C., and later they were transferred to liquid nitrogen for their long-term storage. For the functional assays, PBMCs were thawed in a 37° C. water bath, harvested in RPMI media supplemented with FBS 10% (37° C.), washed twice with culture media and resuspended in culture media. The cellular viability was determined with trypan blue dye.

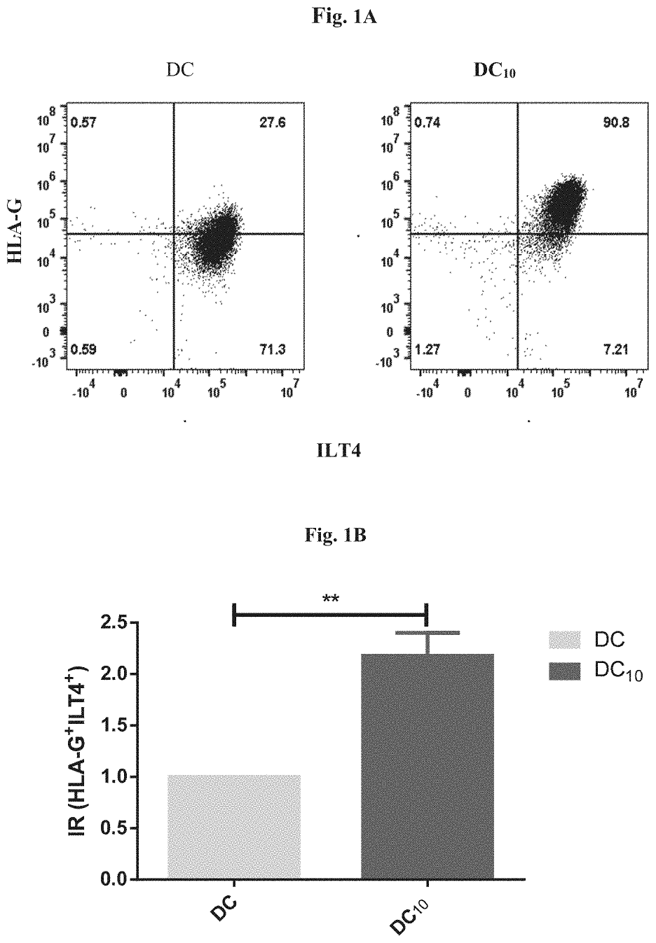

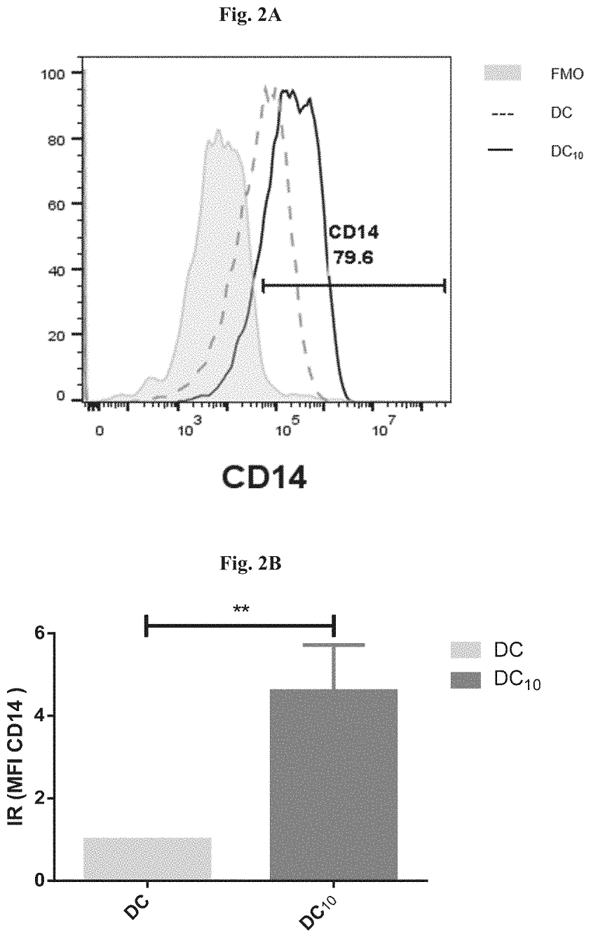

Next, CD14⁺ monocytes were isolated from donor 1 PBMCs by positive selection, using Human CD14 MicroBeads kit (Miltenyi Biotec) following the manufacturer's instructions. Purified CD14⁺ cells were cultured in RPMI media supplemented with 10% HS AB, 50 ng/mL of hrGM-CSF (Peprotech) and 50 ng/mL of rhIL-4 (Peprotech), in the absence (DCs) or presence (DC₁₀) of 10 ng/mL of rhIL-10 (Peprotech), placed into a 48-well plate with 5×10⁵ cells per well. Cells were incubated for 7-8 days, and media with cytokines was exchanged on days 3 and 5. The DCs cultures were activated with 50 mg/ml rhTNF-α (Peprotech) on day 5. Finally, cells were harvested, washed with RPMI media, and resuspended in CTS™ OpTmizer™ T Cell Expansion SFM medium (Gibco) (expansion media), for T cell cocultures. To characterize the DCs, an aliquot of them was stained with anti-HLA-G, anti-ILT4 (FIG. 1) and anti-CD14 antibodies for 20 minutes in the dark, then washed with FACs buffer and acquired in the Attune NxT flow cytometer. Data were analyzed with FlowJo vX.0.0 software (FIG. 2).

### Example 2. Allogeneic Cocultures Between Naïve CD4⁺T Cells and DC₁₀ for Tr1 Lymphocyte Differentiation

To isolate naive CD4+ T cells, 10⁸ PBMCs from donor 2 were incubated with monoclonal antibodies anti-CD4, anti-CD25, and anti CD45RA for 20 min at 4° C. in dark; after that, cells were washed and resuspended in 3 mL of phosphate buffer saline (PBS) 1×. Then naive T cells (CD4+CD25-CD45RA+) were isolated by flow cytometry using FACsAria I (BD Biosciences). Purified cells were harvested in RPMI media supplemented with FBS 20%, centrifuged and washed with PBS 1×. At last, cells were resuspended in PBS 1× and counted for the co-cultures (viability was determined by trypan blue exclusion).

To determine cell proliferation, CD4+CD25−CD45RA+ cells were stained with the vital dye CellTrace™ Violet (CTV, Invitrogen) following the manufacturer's instructions. CD4+CD25−CD45RA+ cells from donor 2 were stained, washed, resuspended in media expansion supplemented with HS-AB 10% and cocultured with DC10 o DCs from example 1 (donor 1) in a proportion of 1:10 (DC:CD4+CD25−CD45RA+ y DC10:CD4+CD25−CD45RA+), using “U” bottom 96-well plates. Cocultures between DC10 and CD4+CD25−CD45RA+ were supplemented with 10 ng/mL of hrIL-10, incubated for 14 days (37° C./5% CO₂) and re-stimulated at day 7 with hrIL-2 (20-50 U/mL) and hrIL-10 (10 ng/mL).

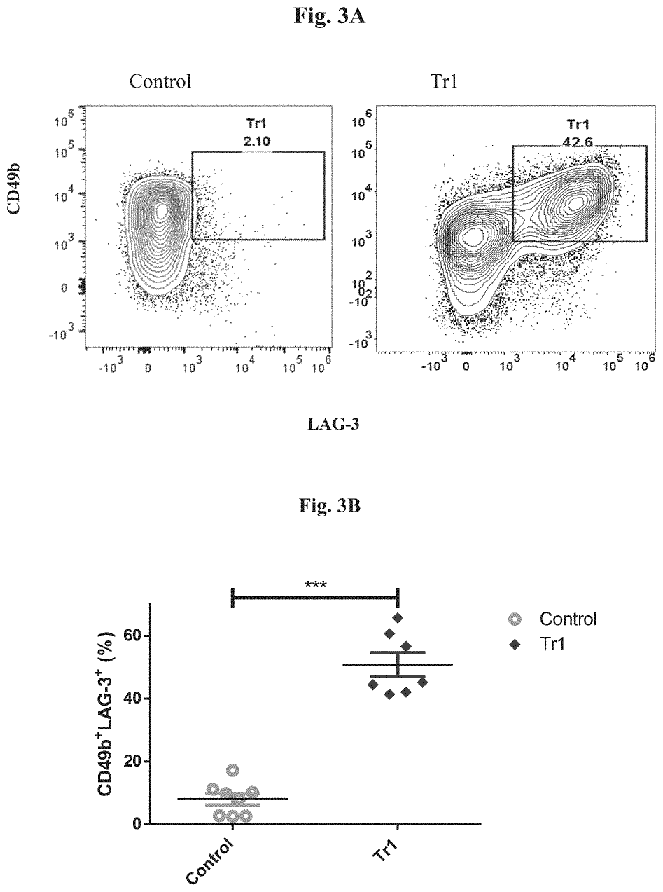

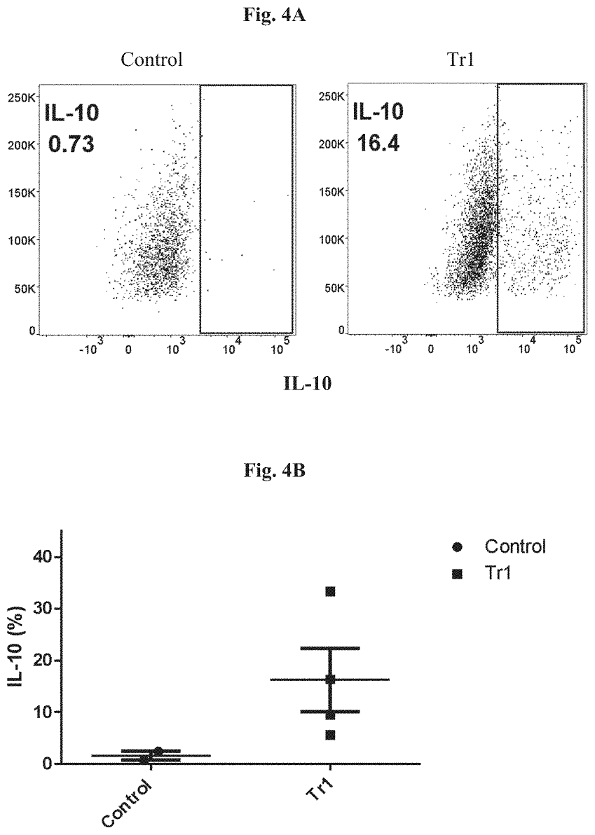

On day 14, cells were stained with the monoclonal antibodies anti-CD4, anti-CD49b, anti-LAG-3 and anti-IL-10, and the samples were acquired in the Attune NxT (Thermo Fisher) cytometer. The percentage of differentiated Tr1 lymphocytes were determined by the co-expression of the surface markers CD49b and LAG-3 (FIG. 3a). In the co-cultures with DC10, a significantly higher percentage of differentiated Tr1 cells was obtained (30-60%), compared with the percentages obtained from the co-cultures with conventional DCs (Control, 2-18%) (FIG. 3b). Moreover, evaluation of IL-10 production (FIG. 4a) from the Tr1 subpopulation (CD49b+LAG-3+), showed a higher proportion of IL-10 producers in the co-cultures with DC10 (10-30%) in comparison to the control group, where production of IL-10 was undetectable (FIG. 4 a-b). Thus, we concluded that under the conditions established in our protocol, it is possible to differentiate high percentages of IL-10 producing allospecific Tr1 lymphocytes, surpassing for the first time, the percentages reported so far using previous methodologies for in vitro Tr1 differentiation (6-12%) proposed for clinical use [24] [25].

### Example 3. Tr1 Purification Based on the Co-Expression of CD49b and LAG-3 Surface Markers

On day 14 of differentiation cultures from example 2, allospecific Tr1 lymphocytes were purified from proliferative cells (CTV−). For this, differentiation co-cultures were stained with anti-CD4, anti-CD49b, anti-LAG-3 monoclonal antibodies, as well as a viability dye, and allospecific Tr1 lymphocytes were purified as live CD4+CD49b+LAG-3+CTV− population by flow cytometry in MoFlo XDP Cell Sorter (Beckman Coulter). Allospecific Tr1 were harvested in RPMI 1640 media supplemented with 20% of FBS and cells were centrifuged discarding the sample supernatant. Tr1 lymphocytes were resuspended in expansion media supplemented with 10% of HS-AB (40-60×10³ per well) in the presence of rhIL-2 (20-50 U/mL) and they were cultured in “U” bottom 96-well plates for 2-3 days. Next, Tr1 lymphocytes were harvested in expansion media, centrifuged and the supernatant was discarded. Then, lymphocytes were resuspended in expansion media supplemented with 10% HS-AB (10-30×10³ cells per well).

### Example 4. Polyclonal Expansion of Purified Tr1 Lymphocytes

Purified Tr1 lymphocytes were stimulated with anti-CD3/anti-CD28 coupled beads (at a proportion 1:5 bead:Tr1), IL-2 (200-250 U/mL) and IL-10 (10 ng/mL). After 4 days of expansion, beads were removed using DynaMag™ (Invitrogen), cells were washed with culture media and cultured again in expansion media with 10% HS-AB supplemented with rhIL-2 (20-50 U/mL) in a “U” bottom 96-well plate for 3 more days (resting phase).

Following this initial expansion scheme, we performed 2-3 more expansion-resting cycles.

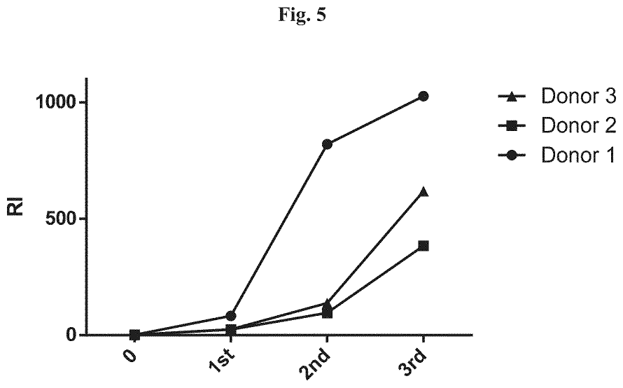

As shown in FIG. 5, under the conditions established in our expansion protocol, allospecific Tr1 cells proliferated in a sustained way along 3 polyclonal expansion cycles, obtaining an increment of up to 1,000× the initial Tr1 numbers. This is relevant because, up until now, no methodologies had yet been developed allowing the sustained expansion of purified Tr1 lymphocytes, to reach the cell numbers required for clinical assays.

### Example 5. Phenotypic Characterization of Purified Expanded Allospecific Tr1 Cells

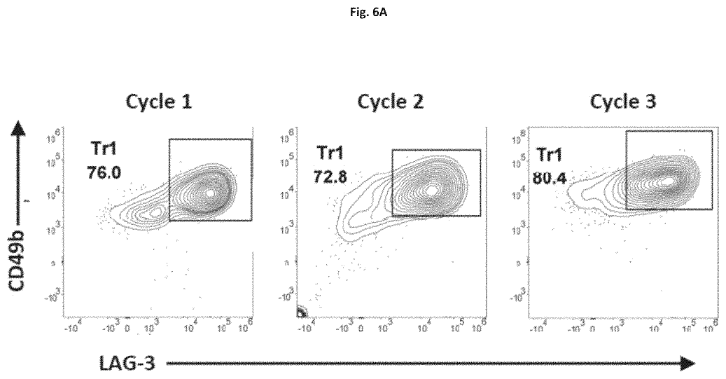

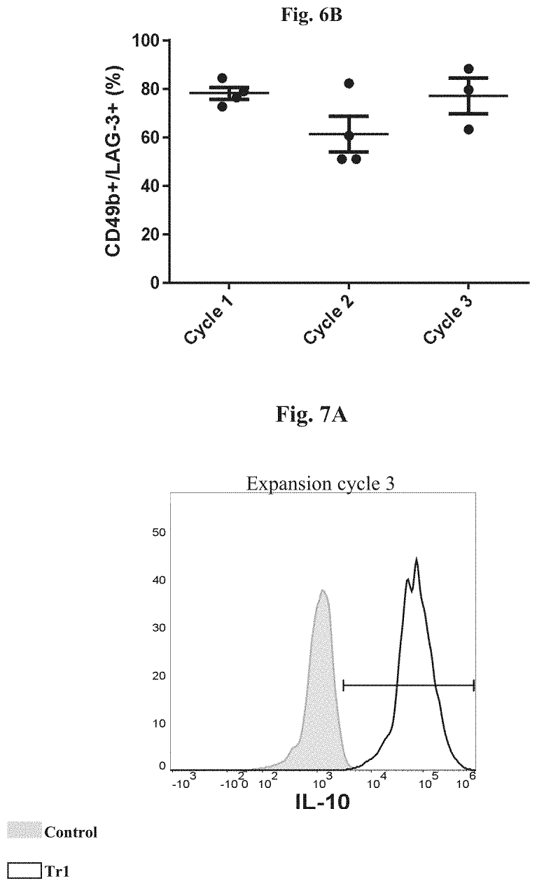

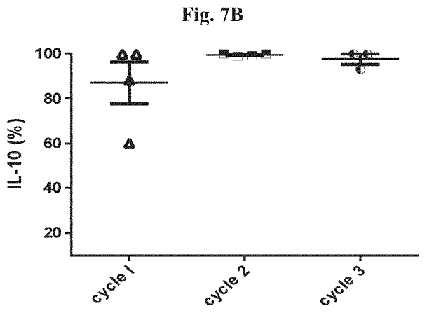

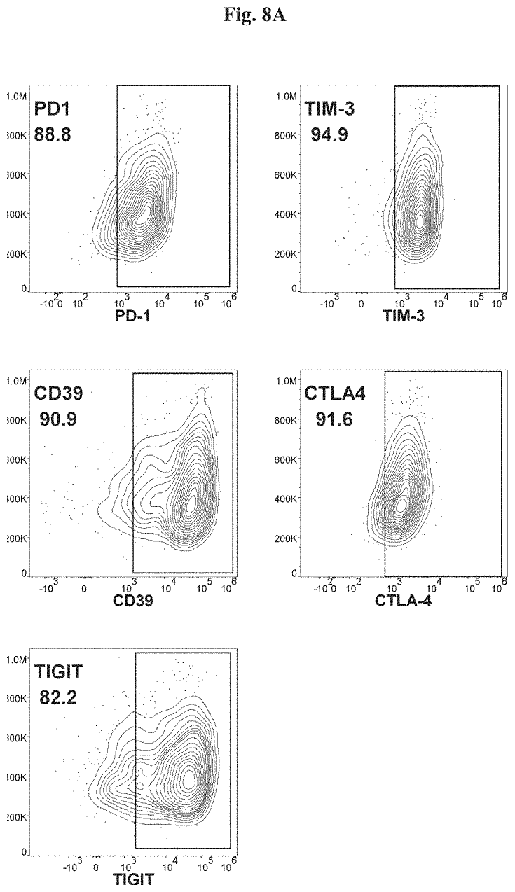

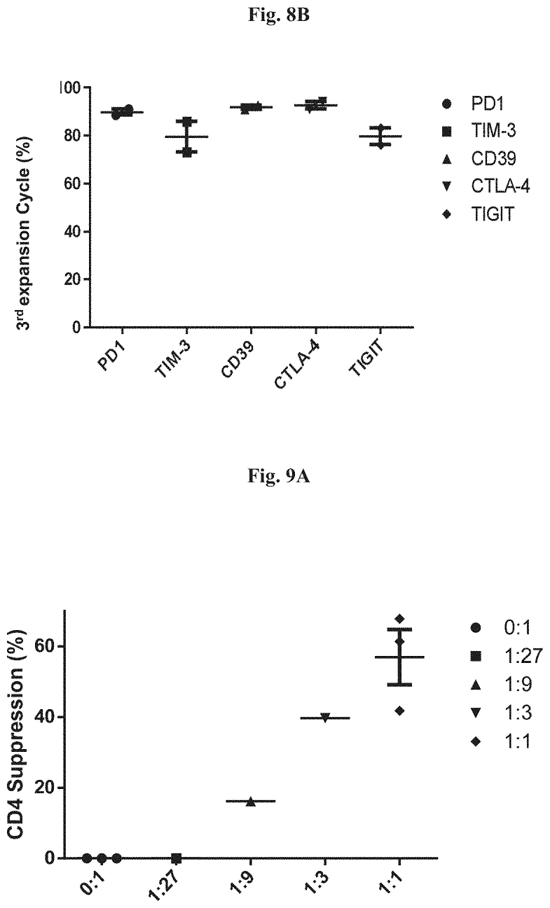

To phenotypically characterize the Tr1 expanded lymphocytes, expression of CD49b, LAG-3, PD-1, TIM-3, CD39, CTLA-4, TIGIT, as well as IL-10 production, were evaluated by flow cytometry. Tr1 lymphocytes maintained a high co-expression of CD49b and LAG-3 molecules (>80%) (FIG. 6). Interestingly, IL-10 production from the differentiated populations described in example 2 were enriched after expansion from a 10% up to >90% (in average) of CD49b+LAG-3+ Tr1 cells (FIG. 7). Finally, our expanded populations expressed the coinhibitory molecules PD1 (88-91%), TIM-3 (73-85.9%), CD39 (90-92%), CTLA-4 (91-94%) y TIGIT (76-84%) (FIG. 7 a-b), which is consistent with the previous reported phenotype corresponding to a Tr1 lymphocyte subpopulation with higher suppressive capacity [28].

In conclusion, our results demonstrate that with the present invention it is possible to isolate and increase the number of allospecific Tr1 lymphocytes with a highly suppressive phenotype.

### Example 6. Functional Characterization of Purified and Expanded Tr1 Cells

To evaluate the function of expanded allospecific Tr1 lymphocytes in vitro suppressive assays were carried out. In these studies DC were differentiated from CD14+ monocytes (allogeneic) from donor 1 and from donor 3, a donor different from donor 1 and 2, following the conditions from example 1. Responder CD3+ T cells (R.C.) were obtained from donor 2 PBMCs, after isolation using the Pan T Cell Isolation Kit (Miltenyi Biotec), following the manufacturer instructions.

The expanded allospecific Tr1 lymphocytes, stained with CTV and R.C. cells, stained with CFSE, were co-cultured in several proportions Tr1:R.C. (0:1, 1:1, 1:3, 1:9, 1:27). The Tr1:R.C. lymphocytes (donor 2) cultures were stimulated with allogeneic DCs (donor 1 or donor 3) in expansion media with 10% HS-AB. On day 4 a surface staining was carried out with anti-CD4, anti-CD8 and anti-CD3 for 20 minutes in the dark, samples were washed with FACS buffer and acquired in the Attune NxT flow cytometer to determine the proliferation of CD8+ and CD4+ T cells by CFSE dilution. To calculate the suppression percentage we used the formula: [(% Tconv proliferation without Tr1−% R.C. in the presence of Tr1)/% R.C. proliferation without Tr1]×100.

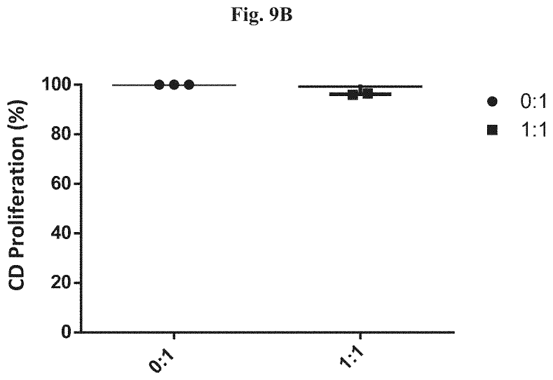

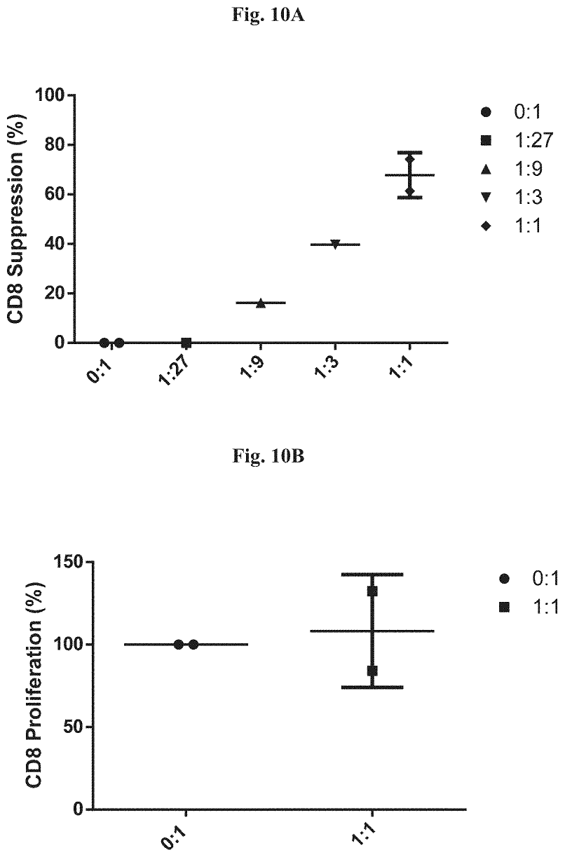

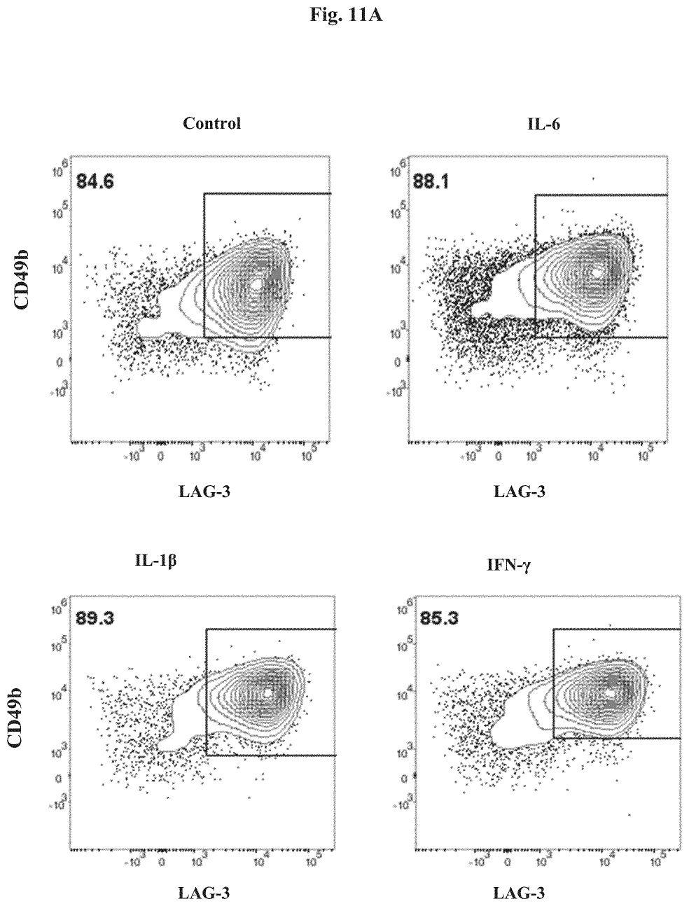

FIGS. 9 and 10 show that Tr1 lymphocytes (donor 2) inhibit the proliferation of Tconv CD4+ (FIG. 9-a) as well as CD8+ (10-a) cells, only when they are stimulated with the DCs used for the initial expansion (donor 1), but do not suppress proliferation in the presence of DCs from a non related donor (third party) (FIG. 9-b y 10-b). In conclusion, Tr1 lymphocytes generated in the present invention suppress the T cell proliferation in an antigen-specific manner.

### Example 7. Evaluation of the Phenotype Stability from Tr1 Lymphocytes Expanded in Presence of Inflammatory Cytokines

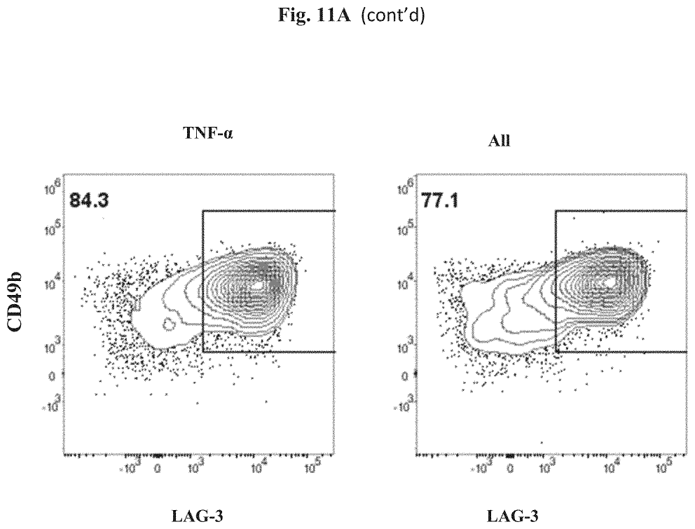

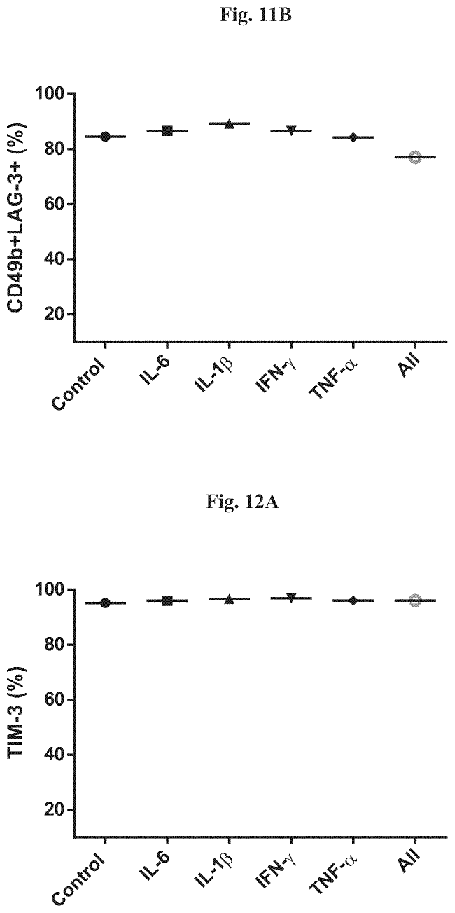

To evaluate the phenotypic stability under an inflammatory microenvironment, allospecific Tr1 lymphocytes, expanded during 3 weeks, were cultured for 2 additional cycles (following the scheme from example 3) in the presence or absence of IL-6 (10 ng/mL), IL-1β (10 ng/mL), IFN-γ (10 ng/mL) and TNF-α (50 ng/mL). At the end of these cycles, expression of CD4, CD49b, LAG-3, TIM-3 and TIGIT was evaluated, together with IL-10 production by flow cytometry. Interestingly, Tr1 lymphocytes maintained the CD49b and LAG-3 (11 a-b) coexpression, as well as co-inhibitory molecules TIM-3 (FIG. 12 a) y TIGIT (FIG. 12 b), after being stimulated in the presence of all the proinflammatory cytokines evaluated. Moreover, IL-10 production is maintained in similar percentages in the presence or absence of proinflammatory cytokines, except for those cells expanded with TNF-α, including the condition with all the cytokines present.

### Example 8. Evaluation of the Suppressive Function Stability from Allospecific Tr1 Lymphocytes Expanded in Presence of Proinflammatory Cytokines

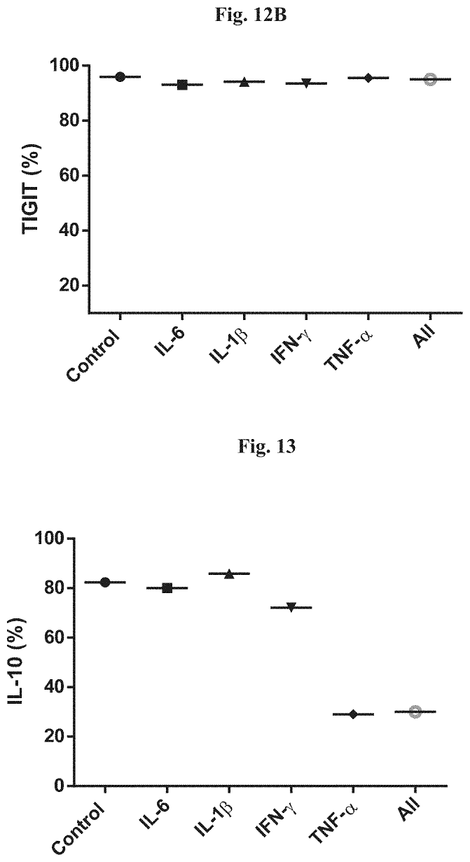

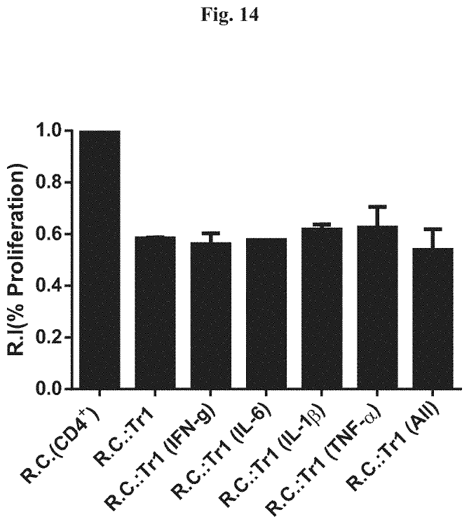

Finally, following the stimulation scheme from Example 5, function stability was evaluated in Tr1 lymphocytes in the presence of proinflammatory cytokines. The results showed that Tr1 cells from donor 2 inhibited the proliferation of alloreactive CD4+ responder T cells (R.C.) at 1:1 ratio (FIG. 14) after being stimulated with inflammatory cytokines, to the same extent as the control condition (without cytokines) despite the decrease in IL-10 caused by TNF-α (FIG. 13 a), demonstrating that Tr1 cells preserve their suppressive capacity even after long term expansion under an inflammatory environment.

Evaluation of phenotypic and functional stability in the presence of inflammatory environments is of vital importance for the cellular products used in transplantation immunotherapy, given the potential inflammatory responses elicited against the graft, in order to provide greater safety to the patients.

### REFERENCES

1. Cosio, F. G., et al., Predicting Subsequent Decline in Kidney Allograft Function from Early Surveillance Biopsies. American Journal of Transplantation, 2005. 5(10): p.
2464-2472.
2. Marcén, R., Immunosuppressive drugs in kidney transplantation: impact on patient survival, and incidence of cardiovascular disease, malignancy and infection. Drugs, 2009. 69(16): p. 2227-43.
3. Hartono, C., T. Muthukumar, and M. Suthanthiran, Immunosuppressive drug therapy. Cold Spring Harb Perspect Med, 2013. 3(9): p. a015487.
4. Denton, M. D., C. C. Magee, and M. H. Sayegh, Immunosuppressive strategies in transplantation. The Lancet, 1999. 353(9158): p. 1083-1091.
5. Atif, M., et al., Regulatory T cells in solid organ transplantation. Clinical & Translational Immunology, 2020. 9(2): p. e01099.
6. Edozie, F. C., et al., Regulatory T-Cell Therapy in the Induction of Transplant Tolerance: The Issue of Subpopulations. Transplantation, 2014. 98(4).
7. Shalev, I., et al., Role of regulatory T cells in the promotion of transplant tolerance. Liver Transplantation, 2012. 18(7): p. 761-770.
8. Vaikunthanathan, T., et al., Regulatory T cells: tolerance induction in solid organ transplantation. Clinical & Experimental Immunology, 2017. 189(2): p. 197-210.
9. Whangbo, J. S., J. H. Antin, and J. Koreth, The role of regulatory T cells in graft-versus-host disease management. Expert Review of Hematology, 2020. 13(2): p. 141-154.
10. Riegel, C., et al., Efficient treatment of murine acute GvHD by in vitro expanded donor regulatory T cells. Leukemia, 2020. 34(3): p. 895-908.
11. Voermans, C. and M. D. Hazenberg, Cellular therapies for graft-versus-host disease: a tale of tissue repair and tolerance. Blood, 2020. 136(4): p. 410-417.
12. BATTAGLIA, M., et al., IL-10-Producing T Regulatory Type 1 Cells and Oral Tolerance. Annals of the New York Academy of Sciences, 2004. 1029(1): p. 142-153.
13. Mowat, A. M., Anatomical basis of tolerance and immunity to intestinal antigens. Nature Reviews Immunology, 2003. 3(4): p. 331-341.
14. Groux, H., et al., A CD4+ T-cell subset inhibits antigen-specific T-cell responses and prevents colitis. Nature, 1997. 389(6652): p. 737-742.
15. Zeng, H., et al., Type 1 regulatory T cells: a new mechanism of peripheral immune tolerance. Cellular & Molecular Immunology, 2015. 12(5): p. 566-571.
16. Levings, M. K., et al., IFN-alpha and IL-10 induce the differentiation of human type 1 T regulatory cells. J Immunol, 2001. 166(9): p. 5530-9.
17. Roncarolo, M. G., et al., The Biology of T Regulatory Type 1 Cells and Their Therapeutic Application in Immune-Mediated Diseases. Immunity, 2018. 49(6): p. 1004-1019.
18. Battaglia, M., et al., Tr1 cells: from discovery to their clinical application. Semin Immunol, 2006. 18(2): p. 120-7.
19. Roncarolo, M. G., et al., Autoreactive T cell clones specific for class I and class II HLA antigens isolated from a human chimera. The Journal of experimental medicine, 1988. 167(5): p. 1523-1534.
20. Vieira, P., et al., Isolation and expression of human cytokine synthesis inhibitory factor cDNA clones: homology to Epstein-Barr virus open reading frame BCRFI. Proceedings of the National Academy of Sciences, 1991. 88(4): p. 1172.
21. Bacchetta, R., et al., High levels of interleukin 10 production in vivo are associated with tolerance in SCID patients transplanted with HLA mismatched hematopoietic stem cells. The Journal of experimental medicine, 1994. 179(2): p. 493-502.
22. Gagliani, N., et al., Coexpression of CD49b and LAG-3 identifies human and mouse T regulatory type 1 cells. Nat Med, 2013. 19(6): p. 739-46.
23. Gregori, S. and M. G. Roncarolo, Engineered T Regulatory Type 1 Cells for Clinical Application. Frontiers in Immunology, 2018. 9(233).
24. Bacchetta, R., et al., Immunological Outcome in Haploidentical-HSC Transplanted Patients Treated with IL-10-Anergized Donor T Cells. Front Immunol, 2014. 5: p. 16.
25. Gregori, S., et al., Differentiation of type 1 T regulatory cells (Tr1) by tolerogenic DC-10 requires the IL-10-dependent ILT4/HLA-G pathway. Blood, 2010. 116(6): p. 935-944.
26. Geissler, E. K., The ONE Study compares cell therapy products in organ transplantation: introduction to a review series on suppressive monocyte-derived cells. Transplantation research, 2012. 1(1): p. 11-11.
27. Mfarrej, B., et al., Generation of donor-specific Tr1 cells to be used after kidney transplantation and definition of the timing of their in vivo infusion in the presence of immunosuppression. J Transl Med, 2017. 15(1): p. 40.
28. Brockmann, L., et al., Molecular and functional heterogeneity of IL-10-producing CD4+ T cells. Nature Communications, 2018. 9(1): p. 5457.

1. A method to generate and expand in vitro allospecific Tr1 lymphocytes comprising the following stages:
a) differentiating tolerogenic Dendritic cells in vitro named DC₁₀ from donor 1;
b) differentiating Allospecific Tr1 by allospecific T naïve (CD4⁺CD25⁻CD45RA⁺) from donor 2 peripheral blood and donor 1 DC₁₀ from the previous stage cocultures;
c) isolating Allospecific Tr1 cell from the previous stage;
d) Polyclonal expanding of isolated allospecific Tr1 cells.

2. The method according to claim 1, wherein DC10 are differentiated from donor 1 peripheral blood CD14⁺ monocytes.

3. The method according to claim 2, wherein more than 60% of allospecific Tr1 cells are obtained during the differentiation stage.

4. The method according to claim 2, wherein the CD14⁺ monocytes are cultured during 7-8 days in presence of recombinant human (rh) GM-CSF (50 ng/mL), rhIL-4 (50 ng/mL) and rhIL-10 (10 ng/mL).

5. The method according to claim 4, wherein a medium refresh is carried out with the cytokines during the days 3 and 5.

6. The method according to claim 1, wherein the allospecific Tr1 cell obtaining of donor 1 DC₁₀ and donor 2 naïve T cells cocultures is carried out by simulating the donor and recipient in the transplantation context, in a 1:5 or a 1:10 ratio respectively.

7. The method according to claim 6, wherein the cell culture is carried out in a presence of hrIL-10 (10 ng/mL) during 7 days of differentiation and restimulating with hrIL-10 (10 ng/mL) and hrIL-2 (20-50 U/mL) during additional 7 days.

8. The method according to claim 1, wherein the isolating of allospecific Tr1 lymphocytes is obtained from CD4⁺CD49b⁺LAG-3⁺CTV⁻ population corresponding to proliferating allospecific cells.

9. The method according to claim 1, wherein the expansion stage is carried out during 3 consecutive cycles, alternating phases of activation and resting.

10. The method according to claim 9, wherein the culturing of Tr1 lymphocytes during the activation stage is carried out in the presence of coupled beads with anti-CD3-CD28 antibodies in a 1:5 (Bead:Tr1) proportion, hrIL-10 (10 ng/mL), and hrIL-2 (200-250 ng/mL) during 4 days.

11. The method according to claim 9, wherein during the resting stage, the polyclonal activation stimulus are removed, and culturing of the Tr1 lymphocytes only in presence of hrIL-2 (20-50 ng/mL) during 3 days is carried out.

12. A Tr1 lymphocytes obtained from the method according to claim 1, wherein the Tr1 lymphocytes maintain 80% of CD49b and LAG-3 and up to 80% of CTLA-4, PD-1, CD39, TIGIT, and TIM-3 co-inhibitory receptors expression.

13. The Tr1 lymphocytes according to claim 12, characterized to be allospecific cells with a high phenotypical and functional stability.

14. The Tr1 lymphocytes according to claim 12, characterized to have up to 80% of purity.

15. The Tr1 lymphocytes according to claim 12, characterized to produce up to 90% of IL-10.

16. A method to generate a cellular product with specific suppressive capacity to be administered by intravenous infusion in pharmacologically effective doses to transplant patients or chronic inflammatory diseases patients, the cellular product comprising the Tr1 lymphocytes according to claim 12.

17. The method according to claim 16, characterized in that the cellular product can be administered as an alternative or complementary treatment to other immunosuppressants.
